(19) United States
(12) Patent Application Publication
(10) Pub. No.: US 2023/0144882 A1
(43) Pub. Date: May 11, 2023

(54) USE OF ADENOSINE DEAMINASE AND ADENOSINEDEAMINASE MODIFIER IN PREPARATION OF MEDICAMENTFOR WOUND REPAIR IN PATIENT WITH DIABETES

(71) Applicant: Nanjing Southern Element Biotechnology Co., Ltd., Nanjing City (CN)

(72) Inventors: Jianfa Zhang, Nanjing City (CN); Tongyuan Lu, Nanjing City (CN)

(21) Appl. No.: 17/982,990

(22) Filed: Nov. 8, 2022

(30) Foreign Application Priority Data
Nov. 10, 2021 (CN) 202111327631.0

(51) Int. Cl. A61K 38/50; A61K 47/60; A61P 3/10; A61P 17/02

(52) U.S. Cl. CPC A61K 38/50; C12Y 305/04004; A61K 47/60; A61P 3/10; A61P 17/02

(57) ABSTRACT

The present disclosure provides use of adenosine deaminase (ADA) and an ADA modifier in preparation of a medicament for wound repair in a patient with diabetes. It is found for the first time that the ADA (EC 3.5.4.4) and a polyethylene glycol-modified adenosine deaminase (PEG-ADA) have a significant improvement effect on wound repair of type-2 diabetic mice, and the ADA or the ADA modifier can be developed into a medicament for treating diabetic wounds.

### CROSS REFERENCE TO RELATED APPLICATION

This patent application claims the benefit and priority of Chinese Patent Application No. 202111327631.0, filed with the China National Intellectual Property Administration on Nov. 10, 2021, the disclosure of which is incorporated by reference herein in its entirety as part of the present application.

### TECHNICAL FIELD

The present disclosure belongs to the technical field of medicine, and relates to use of adenosine deaminase (ADA) and an ADA modifier in preparation of a medicament for wound repair in a patient with diabetes.

### BACKGROUND

About 20% of patients with diabetes experience difficulty in wound repair. Leg or foot ulcers are the most common wounds in patients with diabetes. Diabetic foot caused by the difficulty in diabetic wound repair is the most serious complication and one of the main causes of disability in patients with diabetes. The pathogenesis of diabetic foot is not completely clear. At present, it is believed that disorders of blood lipid and blood glucose metabolisms are closely related to the pathogenesis of diabetic foot. The pathogenesis of diabetic foot is closely related to chronic peripheral vascular disease and peripheral neuropathy. First, patients with diabetes have reduced lower extremity protection due to neuropathy. Second, in patients with diabetes, long-term hyperglycemia leads to arteriosclerosis and develops microcirculation disturbance, ischemia in local tissues, and decreased immunity, and any minor trauma can cause infection and increase ulcers. In patients with diabetes, the glucose metabolism is reduced, and hyperglycemia further complicates the wound repair process, which may lead to chronic stagnation of wound repair. As a result, the course of the disease is prolonged, which brings great pain and economic burden to patients and their families. Therefore, the early treatment of diabetic foot is emphasized to prevent the development of gangrene, which is extremely important to save the affected limb, reduce costs, and improve the quality of life.

Elevation of plasma small molecule adenine nucleotides is a new and important pathological feature of all patients with type 2 diabetes. ADA (EC 3.5.4.4) is a purine decomposition-related catabolic enzyme that converts adenosine to inosine, thereby helping reduce the levels of adenosine present in tissues and cells. Currently, the ADA is often used clinically to detect and characterize some organ and immune diseases, for example, typhoid fever, liver diseases, CAPD-related peritonitis, and severe combined immunodeficiency disease (SCID) (Li X Y, Zhang Z M, Li W. Correlation Research Progress of Determination of Adenosine Deaminase Activity and Clinical Diseases[J]. World Latest Medicine Information, 2018,18(48):28-29.). Polyethylene glycol-modified adenosine deaminase (PEG-ADA) is an enzyme preparation that has been used in a plurality of patients worldwide to detect and treat diseases caused by ADA deficiency such as SCID (Hershfield, M. (2006). Adenosine Deaminase Deficiency. In M. P. Adam (Eds.) et.al., University of Washington, Seattle.). There are no reports on the use of ADA or ADA modifier in the treatment of diabetic wounds.

### SUMMARY

The present disclosure provides use of ADA (EC 3.5.4.4) or an ADA modifier in preparation of a medicament for wound repair in a patient with diabetes.

In the present disclosure, the diabetes includes type 1 and type 2 diabetes, and the ADA and the ADA modifier have more significant effects on the wound repair in a patient with type 2 diabetes.

In the present disclosure, the ADA may be an ADA obtained in any manner, including but not limited to a natural ADA extracted from a biological tissue, a recombinant human-, animal- or microbe-derived ADA, and a chemically synthesized ADA.

Specifically, in a specific embodiment of the present disclosure, the ADA used may be one selected from the group consisting of a naturally extracted bovine adenosine deaminase and an Escherichia coli-expressed murine adenosine deaminase.

In the present disclosure, the ADA modifier may be an ADA modifier obtained by chemically modifying the ADA to increase stability thereof and prolong half-life thereof, including but not limited to a PEG-ADA.

Specifically, in a specific embodiment of the present disclosure, the ADA modifier used may be one selected from the group consisting of a PEG-modified naturally extracted bovine ADA and a PEG-modified E. coli-expressed murine ADA.

In the present disclosure, the medicament for wound repair in a patient with diabetes is a composition containing one or more of the ADA or the ADA modifier, and further contains a pharmaceutically acceptable carrier or vehicle, so that a pharmaceutically acceptable dosage form is prepared.

In the present disclosure, an administration dosage of the ADA or the ADA modifier in the medicament for wound repair in a patient with diabetes provided by the present disclosure may be appropriately adjusted according to the condition. As an optional solution, the ADA and the ADA modifier may have an intraperitoneal injection concentration of 0.1-8 U/g and preferably 5 U/g, and a topical application concentration of 1-300 U/mL and preferably 150 U/mL. (1 U represents a quantity of ADA that decomposes 1 μmol adenosine per minute under specific conditions, U/g represents the activity of ADA injected per gram of patient's body weight, and U/mL represents the activity of the ADA per mL of a solution).

The present disclosure sets forth for the first time that the ADA and the ADA modifier can significantly promote wound repair in diabetic mice. The ADA, as a protein naturally possessed by organisms, has excellent immunogenicity and a wide application prospect.

### BRIEF DESCRIPTION OF THE DRAWINGS

FIG. 1 illustrates an effect of naturally extracted bovine ADA on wound repair in diabetic mice. Compared with wound changes of normal mice (blank control group), diabetic mice and diabetic ADA treatment (injection/dripping) group within 14 days, the diabetic model used is a db/db mouse model of type 2 diabetes, the ADA is the naturally extracted bovine ADA, the injection concentration is 5 U/g, and the dripping concentration is 150 U/mL.

FIG. 2 illustrates an effect of PEG-modified naturally extracted bovine ADA on wound repair in diabetic mice. Compared with wound changes of normal mice (blank control group), diabetic mice and diabetic ADA treatment (injection/dripping) group within 14 days, the diabetic model used is a db/db mouse model of type 2 diabetes, the ADA is the PEG-modified naturally extracted bovine ADA, the injection concentration is 1.5 U/g, and the dripping concentration is 150 U/mL.

FIG. 3 illustrates an effect of E. coli-expressed murine ADA on wound repair in diabetic mice. Compared with wound changes of normal mice (blank control group), diabetic mice and diabetic ADA treatment (injection/dripping) group within 14 days, the diabetic model used is a db/db mouse model of type 2 diabetes induced by streptozotocin (STZ)+high-fat diet, the ADA is the E. coli-expressed murine ADA, the injection concentration is 5 U/g, and the dripping concentration is 150 U/mL.

FIG. 4 illustrates an effect of PEG-modified E. coli-expressed murine ADA on wound repair in diabetic mice. Compared with wound changes of normal mice (blank control group), diabetic mice and diabetic ADA treatment (injection/dripping) group within 14 days, the diabetic model used is a db/db mouse model of type 2 diabetes induced by STZ+high-fat diet, the ADA is the PEG-modified E. coli-expressed murine ADA, the injection concentration is 1.5 U/g, and the dripping concentration is 150 U/mL.

### DETAILED DESCRIPTION OF THE EMBODIMENTS

To make those skilled in the art better understand the solution of the present disclosure, the technical solution of the present disclosure will be described clearly and completely below with reference to the examples of the present disclosure and the accompanying drawings. Apparently, the described examples are only a part of, but not all of, the examples. Based on the examples of the present disclosure, all other examples obtained by those of ordinary skill in the art without creative efforts shall fall within the protection scope of the present disclosure.

All raw materials used in the following examples are commercially available products, unless otherwise specified.

The ADA (EC 3.5.4.4) or the ADA modifier involved in the present disclosure may be purchased or self-prepared.

In the examples, male diabetic mice induced by STZ+high-fat diet and adult male db/db mice are used as models of type 2 diabetes.

### Example 1

Effect of naturally extracted bovine ADA on wound repair in diabetic mice:

1. Experimental Method

1.1 Diabetic Model Establishment:

Db/db mouse model of diabetes: Male db/db diabetic mice (aged 6 weeks) from the Model Animal Research Center, Nanjing University were used in the experiment. All mice were housed under standard raising conditions. The mice were raised under a 12 h:12 h light:dark cycle and had free access to food and water. The mice with fasting blood glucose (FBG) higher than 11.1 mmol/L were considered as type 2 diabetic mice and were selected for subsequent research.

1.2 Establishment of a Mouse Wound Model

Animals in each group were anesthetized with pentobarbital sodium (1%), the back was shaved, and a full-thickness wound with a diameter of 8 mm was cut with scissors at the top of the back. Photographs were taken to record the wound healing of mice after administration, and 1 cm² was used as a scale to analyze the wound healing of the mice in each group.

1.3 Grouping and Administration Method

Blank control group: A mouse wound model was established and treated with the corresponding drug vehicle (phosphate buffered saline, PBS).

Diabetic model group: A mouse model of type 2 diabetes and a mouse wound model were established. The mice were treated with the corresponding drug vehicle (PBS).

Diabetic ADA (injection) treatment group: A mouse model of type 2 diabetes and a mouse wound model were established. Naturally extracted bovine ADA (0.1 U/g, 0.2 U/g, 0.4 U/g, 0.8 U/g, 1.5 U/g, 3 U/g, 5 U/g, and 8 U/g) was intraperitoneally injected daily.

Diabetic ADA (dripping) treatment group: A mouse model of type 2 diabetes and a mouse wound model were established, and bovine ADA (1 U/mL, 2 U/mL, 4 U/mL, 10 U/mL, 30 U/mL, 80 U/mL, 150 U/mL, and 300 U/mL) was dipped on the wound every day.

2. Experimental Results

2.1 Effect of ADA on Wound Healing in Diabetic Mice

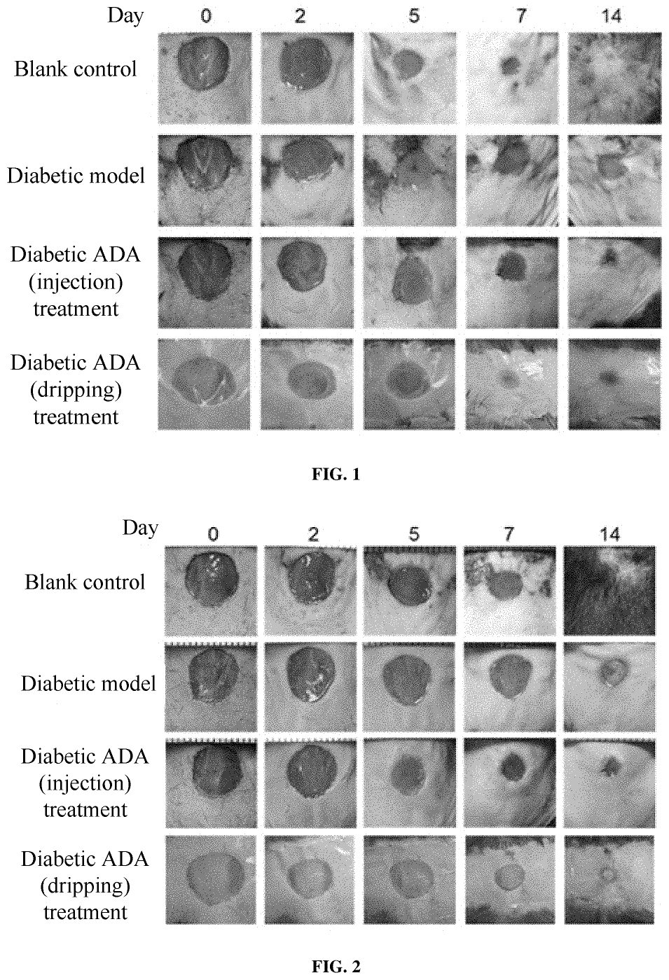

The results are shown in FIG. 1. The wounds in the blank control group healed at about 14 days.

Compared with the blank control group, the wound healing rate of the mice in the diabetic model group was slower, and the wound area was 30±0.5% after 14 days (the percentage represents the wound area at this time point/original wound area, the same below).

Compared with the diabetic model group, the wound healing rate was significantly accelerated and the wound area at 14 days was 10±0.5% in the diabetic ADA (injection) treatment group.

Compared with the diabetic model group, the wound healing rate was significantly accelerated and the wound area at 14 days was 10±0.5% in the diabetic ADA (dripping) treatment group.

The results showed that the naturally extracted bovine ADA could effectively accelerate the wound healing rate of diabetic mice, and the administration by injection was as effective as the administration by dripping.

And, the injection concentration was effective in the range of 0.1 to 8 U/g, and the therapeutic effect was first strong and then weak with the increase of the concentration within the effective concentration range; the optimum concentration was 5 U/g, but the concentration lower than 0.1 U/g or higher than 8 U/g was ineffective. The application and dripping concentration was effective in the range of 1 to 300 U/mL, and the therapeutic effect became stronger at first and then weakened with the increase of the effective concentration within the effective concentration range; the optimum concentration was 150 U/mL, and the concentration lower than 1 U/mL or higher than 300 U/mL was ineffective.

### Example 2

Effect of PEG-modified naturally extracted bovine ADA on wound repair in diabetic mice:

1. Experimental Method

1.1 Preparation of PEG-ADA

ADA was diluted to 500 U/mL with 1 mL of sterile PBS (10 mmol/L, pH 9.0). Subsequently, methoxy polyethylene glycol succinimidyl propionate (mPEG-SPA) with a molecular weight of 20 kDa was added to obtain a final concentration of 100 mg/mL, and the mixture was mixed at room temperature for 5 h to obtain PEG-ADA. Finally, the PEG-ADA was diluted to a final concentration of 150 U/mL with PBS (10 mmol/L, pH 7.4).

1.2 Establishment of a Mouse Model of Diabetes

Db/db mouse model of diabetes: Male db/db diabetic mice (aged 6 weeks) from the Model Animal Research Center, Nanjing University were used in the experiment. All mice were housed under standard raising conditions. The mice were raised under a 12 h:12 h light:dark cycle and had free access to food and water. The mice with FBG higher than 11.1 mmol/L were considered as type 2 diabetic mice and were selected for subsequent research.

1.3 Establishment of a Mouse Wound Model

Animals in each group were anesthetized with pentobarbital sodium (1%), the back was shaved, and a full-thickness wound with a diameter of 8 mm was cut with scissors at the top of the back. Photographs were taken to record the wound healing of mice after administration, and 1 cm² was used as a scale to analyze the wound healing of the mice in each group.

1.4 Grouping and Administration Method

Blank control group: A mouse wound model was established. The mice were treated with the corresponding drug vehicle (PBS).

Diabetic model group: A mouse model of type 2 diabetes and a mouse wound model were established. The mice were treated with the corresponding drug vehicle (PBS).

Diabetic ADA (injection) treatment group: A mouse model of type 2 diabetes and a mouse wound model were established. PEG-modified naturally extracted bovine ADA (0.1 U/g, 0.2 U/g, 0.4 U/g, 0.8 U/g, 1.5 U/g, 3 U/g, 5 U/g, and 8 U/g) was intraperitoneally injected weekly.

Diabetic ADA (dripping) treatment group: A mouse model of type 2 diabetes and a mouse wound model were established, and bovine ADA (1 U/mL, 2 U/mL, 4 U/mL, 10 U/mL, 30 U/mL, 80 U/mL, 150 U/mL, and 300 U/mL) was dipped on the wound every day.

2. Experimental Results

2.1 Effect of ADA on Wound Healing in Diabetic Mice

The results are shown in FIG. 2. The wounds in the blank control group healed at about 14 days.

Compared with the blank control group, the wound healing rate of the mice in the diabetic model group was slower, and the wound area was 30±0.5% after 14 days.

Compared with the diabetic model group, the wound healing rate was significantly accelerated and the wound area at 14 days was 10±0.5% in the diabetic ADA (injection) treatment group.

Compared with the diabetic model group, the wound healing rate was significantly accelerated and the wound area at 14 days was 10±0.5% in the diabetic ADA (dripping) treatment group.

The results showed that the PEG-modified naturally extracted bovine ADA could effectively accelerate the wound healing rate of diabetic mice, and the administration by injection was as effective as the administration by dripping.

And, the injection concentration was effective in the range of 0.1 to 8 U/g, and the therapeutic effect was first strong and then weak with the increase of the concentration within the effective concentration range; the optimum concentration was 1.5 U/g, but the concentration lower than 0.1 U/g or higher than 8 U/g was ineffective. The application and dripping concentration was effective in the range of 1 to 300 U/mL, and the therapeutic effect became stronger at first and then weakened with the increase of the effective concentration within the effective concentration range; the optimum concentration was 150 U/mL, and the concentration lower than 1 U/mL or higher than 300 U/mL was ineffective.

### Example 3

Effects of E. coli-expressed murine ADA on wound repair in diabetic mice:

1. Experimental Method

1.1 For the preparation of E. coli-expressed murine ADA, refer to the corresponding literature [Kim D, Ku S. Bacillus Cellulase Molecular Cloning, Expression, and Surface Display on the Outer Membrane of Escherichia coli. Molecules. 2018;23(2):503. Published 2018 Feb. 24. doi: 10.3390/molecules23020503].

1.2 Establishment of a Mouse Model of Diabetes

Type 2 diabetic model induced by STZ+high-fat diet: Male C57BL/6 mice (aged 8-10 weeks) from the Model Animal Research Center, Nanjing University were used in the experiment. All mice were housed under standard raising conditions. The mice were raised under a 12 h:12 h light:dark cycle and had free access to food and water. After four-week feeding, intraperitoneal injection was induced with 30 mg/kg STZ for three consecutive days. The mice with FBG higher than 11.1 mmol/L were considered as type 2 diabetic mice and were selected for subsequent research.

1.3 Establishment of a Mouse Wound Model

Animals in each group were anesthetized with pentobarbital sodium (1%), the back was shaved, and a full-thickness wound with a diameter of 8 mm was cut with scissors at the top of the back. Photographs were taken to record the wound healing of mice after administration, and 1 cm² was used as a scale to analyze the wound healing of the mice in each group.

1.4 Grouping and Administration Method

Blank control group: A mouse wound model was established. The mice were treated with the corresponding drug vehicle (PBS).

Diabetic model group: A type 2 diabetic mouse wound model was established. The mice were treated with the corresponding drug vehicle (PBS).

Diabetic ADA (injection) treatment group: A mouse model of type 2 diabetes and a mouse wound model were established. Murine ADA (0.1 U/g, 0.2 U/g, 0.4 U/g, 0.8 U/g, 1.5 U/g, 3 U/g, 5 U/g, and 8 U/g) was intraperitoneally injected weekly.

Diabetic ADA (dripping) treatment group: A mouse model of type 2 diabetes and a mouse wound model were established, and murine ADA (1 U/mL, 2 U/mL, 4 U/mL, 10 U/mL, 30 U/mL, 80 U/mL, 150 U/mL, and 300 U/mL) was dipped on the wound every day.

2. Experimental Results

2.1 Effect of ADA on Wound Healing in Diabetic Mice

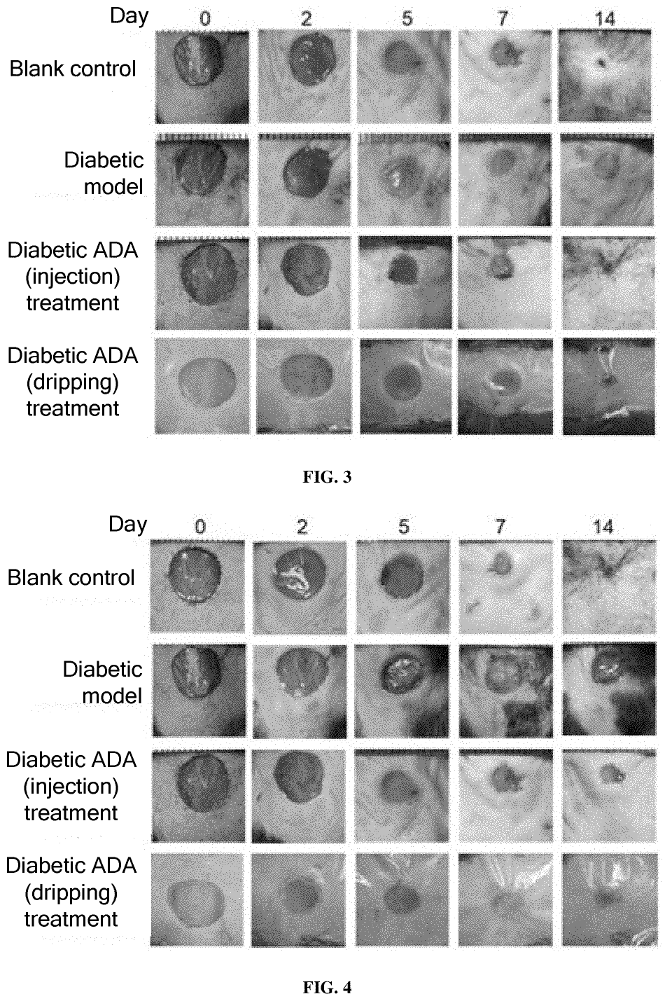

The results are shown in FIG. 3. The wounds in the blank control group healed at about 14 days.

Compared with the blank control group, the wound healing rate of the mice in the diabetic model group was slower, and the wound area was still 30±0.5% after 14 days.

Compared with the diabetic model group, the wound healing rate was significantly accelerated and the wound area at 14 days was 10±0.5% in the diabetic ADA (injection) treatment group.

Compared with the diabetic model group, the wound healing rate was significantly accelerated and the wound area at 14 days was 10±0.5% in the diabetic ADA (dripping) treatment group.

The results showed that the E. coli-expressed murine ADA could effectively accelerate the wound healing rate of diabetic mice, and the administration by injection was as effective as the administration by dripping.

And, the injection concentration was effective in the range of 0.1 to 8 U/g, and the therapeutic effect was first strong and then weak with the increase of the concentration within the effective concentration range; the optimum concentration was 5 U/g, but the concentration lower than 0.1 U/g or higher than 8 U/g was ineffective. The application and dripping concentration was effective in the range of 1 to 300 U/mL, and the therapeutic effect became stronger at first and then weakened with the increase of the effective concentration within the effective concentration range; the optimum concentration was 150 U/mL, and the concentration lower than 1 U/mL or higher than 300 U/mL was ineffective.

### Example 4

Effect of PEG-modified E. coli-expressed murine ADA on wound repair in diabetic mice:

1. Experimental Method

1.1 For the preparation of E. coli-expressed murine ADA, refer to the corresponding literature [Kim D, Ku S. Bacillus Cellulase Molecular Cloning, Expression, and Surface Display on the Outer Membrane of Escherichia coli. Molecules. 2018;23(2):503. Published 2018 Feb. 24. doi: 10.3390/molecules23020503].

1.2 Preparation of PEG-Modified ADA

ADA was diluted to 500 U/mL with 1 mL of sterile PBS (10 mmol/L, pH 9.0). Subsequently, mPEG-SPA with a molecular weight of 20 kDa was added to obtain a final concentration of 100 mg/mL, and the mixture was mixed at room temperature for 5 h. Finally, the PEG-ADA was diluted to a final concentration of 150 U/mL with PBS (10 mmol/L, pH 7.4).

1.3 Establishment of a Mouse Model of Diabetes

Type 2 diabetic model induced by STZ+high-fat diet: Male C57BL/6 mice (aged 8-10 weeks) from the Model Animal Research Center, Nanjing University were used in the experiment. All mice were housed under standard raising conditions. The mice were raised under a 12 h:12 h light:dark cycle and had free access to food and water. After four-week feeding, intraperitoneal injection was induced with 30 mg/kg STZ for three consecutive days. The mice with FBG higher than 11.1 mmol/L were considered as type 2 diabetic mice and were selected for subsequent research.

1.4 Establishment of a Mouse Wound Model

Animals in each group were anesthetized with pentobarbital sodium (1%), the back was shaved, and a full-thickness wound with a diameter of 8 mm was cut with scissors at the top of the back. Photographs were taken to record the wound healing of mice after administration, and 1 cm² was used as a scale to analyze the wound healing of the mice in each group.

1.5 Grouping and Administration Method

Blank control group: A mouse wound model was established. The mice were treated with the corresponding drug vehicle (PBS).

Diabetic model group: A type 2 diabetic mouse wound model was established. The mice were treated with the corresponding drug vehicle (PBS).

Diabetic ADA (injection) treatment group: A mouse model of diabetes and a mouse wound model were established. E. coli-expressed murine ADA (0.1 U/g, 0.2 U/g, 0.4 U/g, 0.8 U/g, 1.5 U/g, 3 U/g, 5 U/g, and 8 U/g) was intraperitoneally injected weekly.

Diabetic ADA (dripping) treatment group: A db/db mouse model of diabetes and a mouse wound model were established, and PEG-modified murine ADA (1 U/mL, 2 U/mL, 4 U/mL, 10 U/mL, 30 U/mL, 80 U/mL, 150 U/mL, and 300 U/mL) was dipped on the wound every day.

2. Experimental Results

2.1 Effect of ADA on Wound Healing in Diabetic Mice

The results are shown in FIG. 4. The wounds in the blank control group healed at about 14 days.

Compared with the blank control group, the wound healing rate of the mice in the diabetic model group was slower, and the wound area was still 30±0.5% after 14 days.

Compared with the diabetic model group, the wound healing rate was significantly accelerated and the wound area at 14 days was 10±0.5% in the diabetic ADA (injection) treatment group.

Compared with the diabetic model group, the wound healing rate was significantly accelerated and the wound area at 14 days was 10±0.5% in the diabetic ADA (dripping) treatment group.

The results showed that the PEG-modified E. coli-expressed murine ADA could effectively accelerate the wound healing rate of diabetic mice, and the administration by injection was as effective as the administration by dripping.

And, the injection concentration was effective in the range of 0.1 to 8 U/g, and the therapeutic effect was first strong and then weak with the increase of the concentration within the effective concentration range; the optimum concentration was 1.5 U/g, but the concentration lower than 0.1 U/g or higher than 8 U/g was ineffective. The application and dripping concentration was effective in the range of 1 to 300 U/mL, and the therapeutic effect became stronger at first and then weakened with the increase of the effective concentration within the effective concentration range; the optimum concentration was 150 U/mL, and the concentration lower than 1 U/mL or higher than 300 U/mL was ineffective.

What is claimed is:

1. A method for wound repair in a patient with diabetes, wherein adenosine deaminase (ADA) (EC 3.5.4.4) or an ADA modifier is used in the wound repair in a patient with diabetes.

2. The method for wound repair in a patient with diabetes according to claim 1, wherein the diabetes is type 1 or type 2 diabetes.

3. The method for wound repair in a patient with diabetes according to claim 1, wherein the ADA is one selected from the group consisting of a natural ADA extracted from a biological tissue, a recombinant human-, animal- or microbe-derived ADA, and a chemically synthesized ADA.

4. The method for wound repair in a patient with diabetes according to claim 1, wherein the ADA is one selected from the group consisting of a naturally extracted bovine adenosine deaminase and an Escherichia coli-expressed murine adenosine deaminase.

5. The method for wound repair in a patient with diabetes according to claim 1, wherein the ADA modifier is an ADA modifier obtained by chemically modifying the ADA to increase stability thereof and prolong half-life thereof.

6. The method for wound repair in a patient with diabetes according to claim 1, wherein the ADA modifier is a polyethylene glycol-modified adenosine deaminase (PEG-ADA).

7. The method for wound repair in a patient with diabetes according to claim 6, wherein the PEG-ADA is one selected from the group consisting of a PEG-modified naturally extracted bovine ADA and a PEG-modified Escherichia coli-expressed murine ADA.

8. The method for wound repair in a patient with diabetes according to claim 1, wherein the medicament for wound repair in a patient with diabetes is a composition comprising one or more of the ADA or the ADA modifier, and further comprises a pharmaceutically acceptable carrier or vehicle.

9. The method for wound repair in a patient with diabetes according to claim 1, wherein the ADA and the ADA modifier have an intraperitoneal injection concentration of 0.1-8 U/g, and a topical application concentration of 1-300 U/mL.

10. The method for wound repair in a patient with diabetes according to claim 1, wherein the ADA and the ADA modifier have an intraperitoneal injection concentration of 5 U/g, and a topical application concentration of 150 U/mL.
